(19) United States
(12) Patent Application Publication
(10) Pub. No.: US 2003/0040461 A1
(43) Pub. Date: Feb. 27, 2003

(54) Modulators of Bruton'sTyrosine Kinase and Bruton's Tyrosine Kinase intermediates and methods for their identification and use in the treatment and prevention of osteoporosis and related diseases states

(76) Inventor: C. Patrick McAtee, Pennington, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/045,202

(22) Filed: Oct. 22, 2001

Related U.S. Application Data
(60) Provisional application No. 60/242,471, filed on Oct. 23, 2000.

(51) Int. Cl. A61K 31/00; C12Q 1/48

(52) U.S. Cl. 514/1; 435/15

(57) ABSTRACT

The present invention relates to the identification of Bruton's Tyrosine Kinase as a critical intermediate in the process of osteoclast activation, modulators of Bruton's Tyrosine Kinase, and assays for the identification of such modulators. It is now found that such modulators are useful in the treatment and prevention of osteoporosis and related disease states.

### CROSS-REFERENCE TO RELATED APPLICATIONS

This application claims priority to U.S. Provisional Patent Application No. 60/242,471, filed Oct. 23, 2000, and hereby expressly incorporated by reference in its entirety.

### FIELD OF THE INVENTION

The present invention relates to kinase modulators and methods for their identification and use in the treatment and prevention of disease. Particularly, the present invention relates to modulators of Bruton's Tyrosine Kinase and Bruton's Tyrosine Kinase intermediates and methods for their identification and use in the treatment and prevention of osteoporosis and related disease states.

### BACKGROUND OF RELATED TECHNOLOGY

The osteoclast is a terminally differentiated cell derived from monocytic/macrophage lineage which resorbs bone as part of the normal process of skeletal modeling and remodeling. In contrast to precursor cells, only fully differentiated mature osteoclasts are able to resorb bone. Increased osteoclastic bone resorption has been linked to the pathogenesis of several skeletal disorders, most notably post-menopausal osteoporosis.

As activated osteoclasts move over the bone surface to initiate new sites of bone resorption, cytoskeletal rearrangements lead to the formation of unique cell adhesion structures called podosomes, which attach to the bone matrix via intermediate steps. Podosomes consist of an F-actin core surrounded by the actin-binding proteins vinculin, talin, and α-actinin, and are found in a variety of highly motile cells such as monocytes or macrophages. (Marchisio P. C., et al., J. Cell Biol. 99(5):1696-1705 (1984)). Podosome assembly is essential to formation of the sealing zone between osteoclasts and the bone matrix, and subsequent bone resorption by the osteoclast is dependent upon the formation of the sealing zone.

It is known that osteoclast precursor cells possess a receptor, receptor activator of NF-κB (RANK), that recognizes a ligand (RANKL) which leads to osteoclast differentiation. (Suda, T., et al.. Endocr. Rev., 20:345-357 (1999)). The RANKL receptor is a member of the tumor necrosis factor (TNF) family and has previously been shown to be an activator of NF-κB and is a specific inducer of osteoclastogenesis. (Simonet W. S., et. al., Cell 89(2):309-319 (1997); Kong Y. Y. et. al., Nature 397(6717):315-323 (1999)).

Although PI3kinase, rhoA, and pp60c-src have been shown to be essential for cytoskeletal rearrangement and osteoclast mediated bone resorption, little is known of the signal transduction events initiated through the RANKL receptor. (Nakamura I., et al., J. Cell Physiol. 172(2):230-239 (1997); Chellaiah M. A., et al., J. Biol. Chem. 275(16):11993-20002 (2000); Schwartzberg P. L., et al., Genes Dev. 11(21):2835-44 (1997)). Several components of the PI3 kinase heteromultimeric complex have been reported to be responsible for osteoclast activation and bone resorption. In previous studies, it has been shown that RANKL is a key regulator of osteoclastogenesis and that the PI3 kinase complex is associated with the RANKL receptor. While it has been reported that PI3 kinase is involved with ruffled border formation in osteoclasts and that wormannin, a PI3 kinase inhibitor, will affect osteoclast attachment and spreading leading to subsequent osteopenia, the involvement of BTK in this process has not been previously demonstrated.

Additionally, other kinases have been reported to play a role in osteoclast activation. (Matsumoto M., et al., J. Biol. Chem. 275, (40) 31155-61 (2000). However, the link between these kinases, RANKL, and cytoskeletal reorganization during the activation cycle remains largely unidentified.

Accordingly, there exists a continuing need to identify compounds involved in the RANKL pathway, as well as modulators thereof, which are useful for the identification, prevention and treatment of osteoporosis, related disease states and other diseases. The present invention is directed towards meeting these and other needs.

### SUMMARY OF THE INVENTION

It has now been found that Bruton's Tyrosine Kinase (BTK) and intermediates in the BTK pathway are critical intermediates in the cytoskeletal rearrangement pathway leading to osteoclast activation. The present invention further shows that mice deficient in BTK exhibit osteopenia and that this osteopenia can be reversed upon the addition of multiple copies of the BTK gene in transgenic mice. Accordingly, modulators of BTK activity and BTK intermediate activity are useful in affecting osteoclast activation and bone resorption. Such modulators may be identified using assays of the present invention, and are therefore expected to be useful as therapeutic compounds to treat osteoporosis and related disease states. BTK target validation studies on modulators identified using methods of the present invention may be carried out using conventional osteoporosis mouse models. Further, such compounds are suitable for use in compositions for the treatment of osteoporosis and related disease states, and may be administered in any conventional manner. The present invention further includes the use of antisense therapy.

In one aspect, the present invention is directed to an assay for identifying a compound that modulates the activity of BTK. This assay includes the steps of: (1) providing a cell expressing BTK; (2) contacting the cell expressing BTK with a test compound; and (3) determining whether the test compound modulates the activity of BTK. This assay may be a cell-based assay or may be a cell-free assay, such as a ligand-binding assay. Test compounds which modulate the activity of BTK may be antagonists or agonists, and may bind to BTK. Further, this assay may be used for identifying compounds which will be useful for the treatment of osteoporosis.

In another aspect, the present invention is directed to a method for the treatment of osteoporosis, which includes the step of administering to a patient in need thereof a therapeutically effective amount of a compound identified by the above assay.

In another aspect, the present invention is directed to a method for the treatment of osteoporosis, which includes the steps of: (1) identifying a patient suffering from osteoporosis; and (2) administering to the patient a therapeutically effective amount of a modulator of BTK. The patient may be identified as suffering from osteoporosis by measuring the expression level of BTK in the patient.

In another aspect, the present invention is directed to a method for the prevention of osteoporosis. This method includes the steps of: (1) identifying a patient at risk for osteoporosis; and (2) administering to the patient a therapeutically effective amount of a modulator of BTK. The patient may be identified as being at risk for osteoporosis by measuring the expression level of BTK in the patient.

In another aspect, the present invention is directed to a method of decreasing the differentiation of osteoclast precursor cells into osteoclast cells. This method includes the step of contacting the osteoclast precursor cells with a BTK modulator.

In another aspect, the present invention is directed to a compound capable of modulating the activity of BTK. This compound may be identified by the steps of: (1) providing a cell expressing BTK; (2) contacting the cell expressing BTK with the compound; and (3) determining whether the compound modulates the activity of BTK. Such a compound may bind to BTK.

### BRIEF DESCRIPTION OF THE DRAWINGS

FIG. 1 shows bone mineral density results for BTK knockout versus wild-type mice.

FIG. 2 shows bone mineral density results for BTK^{xid} mice versus wild-type mice.

FIG. 3 shows the bone mineral density for female BTK^{xid} mice versus wild-type mice with the addition of one and two copies of wild-type BTK on the BTK^{xid} background.

FIG. 4 shows a summary of molecular constructs generated for studying BTK.

FIG. 5 shows a one-dimensional Western blot showing the detection of FLAG BTK in transfected COS-7 and HEK 293 lysates.

FIG. 6 shows a one-dimensional Western blot showing the detection of FLAG BTK in transfected stable RAW 264.7 cell lysates.

FIGS. 7a and 7b show one-dimensional Western blots showing wild-type BTK and mutant BTK phosphorylation.

FIG. 7c shows fluorometric densitometry analysis of antiflag fluorescence versus anti phosphotyrosine fluorescence for FLAG tagged BTK and mutants.

FIG. 8 shows a one-dimensional Western blot showing wild-type BTK and mutant BTK total cellular tyrosine phosphorylation.

FIG. 9 shows immunoprecipitation and kinase assays using SLP 76 as a kinase substrate.

FIGS. 10a, 10b and 10c show actin phalloidin staining of BTK mutant transfected stable RAW 264.7 cell lines.

### DETAILED DESCRIPTION OF THE INVENTION

The osteoclast is a terminally differentiated cell derived from monocytic/macrophage lineage that resorbs bone as part of the normal process of skeletal remodeling. Increased osteoclastic bone resorption leads to many skeletal disorders, most notably post-menopausal osteoporosis in adult women and frailty in adult men. Through development of podosomes, activated osteoclasts move over the bone surface to initiate new sites of bone resorption. These events are initiated preferentially through the interaction of receptor activator of NF-κB ligand (RANKL) with the RANKL receptor present on the osteoclast membrane. RAW 264.7 cells may be differentiated into functional osteoclasts upon activation with RANKL. The present invention is directed to the finding that these cells and osteoclasts derived from human and mouse bone tissue have been found to express BTK.

As set forth hereinbelow, in the present invention BTK^{-/-} (Petro J. B., et al., J. Exp. Med. 191(10): 1745-1754) mice proximal tibia sections evaluated for bone mineral density by peripheral quantitation computed tomography show evidence of osteopetrosis compared to wild-type mice. On the other hand, BTK^{xid} mice (Pinschewer D. D., et al., Eur. J. Immunol. 29(9):2981-2987), wherein the mutation results in a conversion of arginine to cysteine at residue 28, are found in the present invention to be osteoporotic compared to wild-type mice. As a result of this mutation, the BTK protein is unable to translocate from the cytosol to the inner cell membrane where it subsequently binds to the phospholipid product of PI3 kinase, PIP₃.

Following binding to the phospholipid moiety through the pleckstrin homology domain, BTK is phosphorylated by a membrane associated src protein which activates BTK. The activated BTK may then translocate to other subcellular compartments and subsequently regulate other cellular pathways through either its enzymatic activity or association with other regulatory or structural proteins. The osteoporotic effect seen in BTK^{xid} mice is reversed by the addition of copies of wild-type BTK transgenes into the BTK/xid background. Accordingly, these results of the present invention show that BTK is a critical enzyme in the process of bone resorption and clinical osteoporosis.

Analysis of stable BTK constructs expressed on a RAW 264.7 cell background determined that autophosphorylation of BTK may be inhibited through either the xid mutation or the BTK dominant negative mutation (substitution of arginine for lysine at residue 430 in the kinase domain). However, as opposed to the dominant negative mutant as well as the other constructs, the kinase activity of BTK isolated from the xid mutant stable cell pool was significantly higher (approximately 10 fold). Immunofluroescence observations of BTK stable RAW 264.7 cell pools indicated the following differences between mutants stained with actin/phalloidin staining: The dominant negative mutant contained a single ring of podosomes with some stress fibers and cytoplasmic staining; the xid mutant contained a double ring of podosomes, irregularly shaped cells with large sealing zones, and cytoplasmic staining; and the “gain of function” mutation (Li T., et al., Immunity. 2(5):451-460) (lysine substituted for glutamic acid at residue 41 in the pleckstrin homology domain) yielded numerous large cells containing a significant amount of stress fibers, reminiscent of cytoskeletal changes observed in RAW 264.7 cells following activation with osteopontin. BTK antibody staining showed localization at or near the membrane regardless of the mutation. These studies of the present invention establish BTK and subsequent downstream effectors as critical to podosome assembly and, accordingly, osteoclast activation and development of osteopenia.

The reported DNA sequence (SEQ ID NO: 1) and amino acid sequence (SEQ ID NO:2) of human BTK is set forth in Tables 1 and 2, below, respectively. The reported DNA sequence (SEQ ID NO:3) and amino acid sequence (SEQ ID NO:4) of murine BTK is set forth in Tables 3 and 4, below, respectively. Both human and murine BTK sequences were obtained from the Genbank database.

One of skill in the art will recognize that BTK suitable for use in the present invention is desirably murine or human, but may include BTK from any suitable organism. The protein and genomic sequences of these organisms are readily accessed via Genbank or The National Center for Biotechnology Information.

TABLE 1
Nucleotide Sequence of Human BTK: Genbank Accession No. X58957

```
      1 cgtatgtctc cagggccagt gtctgctgcg atcgagtccc accttccaag tcctggcatc
    61 tcaatgcatc tgggaagcta cctgcattaa gtcaggactg agcacacagg tgaactccag
  121 aaagaagaag ctatggccgc agtgattctg gagagcatct ttctgaagcg atcccaacag
  181 aaaaagaaaa catcacctct aaacttcaag aagcgcctgt ttctcttgac cgtgcacaaa
  241 ctctcctact atgagtatga ctttgaacgt gggagaagag gcagtaagaa gggttcaata
  301 gatgttgaga agatcacttg tgttgaaaca gtggttcctg aaaaaaatcc tcctccagaa
  361 agacagattc cgagaagagg tgaagagtcc agtgaaatgg agcaaatttc aatcattgaa
  421 aggttccctt atcccttcca ggttgtatat gatgaagggc ctctctacgt cttctcccca
  481 actgaagaac taaggaagcg gtggattcac cagctcaaaa acgtaatccg gtacaacagt
  541 gatctggttc agaaatatca cccttgcttc tggatcgatg ggcagtatct ctgctgctct
  601 cagacagcca aaaatgctat gggctgccaa attttggaga acaggaatgg aagcttaaaa
  661 cctgggagtt ctcaccggaa gacaaaaaag cctcttcccc caacgcctga ggaggaccag
  721 atcttgaaaa agccactacc gcctgagcca gcagcagcac cagtctccac aagtgagctg
  781 aaaaaggttg tggcccttta tgattacatg ccaatgaatg caaatgatct acagctgcgg
  841 aagggtgatg aatattttat cttggaggaa agcaacttac catggtggag agcacgagat
  901 aaaaatgggc aggaaggcta cattcctagt aactatgtca ctgaagcaga agactccata
  961 gaaatgtatg agtggtattc caaacacatg actcggagtc aggctgagca actgctaaag
1021 caagagggga aagaaggagg tttcattgtc agagactcca gcaaagctgg caaatataca
1081 gtgtctgtgt ttgctaaatc cacaggggac cctcaagggg tgatacgtca ttatgttgtg
1141 tgttccacac ctcagagcca gtattacctg gctgagaagc accttttcag caccatccct
1201 gagctcatta actaccatca gcacaactct gcaggactca tatccaggct caaatatcca
1261 gtgtctcaac aaaacaagaa tgcaccttcc actgcaggcc tgggatacgg atcatgggaa
1321 attgatccaa aggacctgac cttcttgaag gagctgggga ctggacaatt tggggtagtg
1381 aagtatggga aatggagagg ccagtacgac gtggccatca agatgatcaa agaaggctcc
1441 atgtctgaag atgaattcat tgaagaagcc aaagtcatga tgaatctttc ccatgagaag
1501 ctggtgcagt tgtatggcgt ctgcaccaag cagcgcccca tcttcatcat cactgagtac
1561 atggccaatg gctgcctcct gaactacctg agggagatgc gccaccgctt ccagactcag
1621 cagctgctag agatgtgcaa ggatgtctgt gaagccatgg aatacctgga gtcaaagcag
1681 ttccttcacc gagacctggc agctcgaaac tgtttggtaa acgatcaagg agttgttaaa
1741 gtatctgatt tcggcctgtc caggtatgtc ctggatgatg aatacacaag ctcagtaggc
1801 tccaaatttc cagtccggtg gtccccaccg gaagtcctga tgtatagcaa gttcagcagc
1861 aaatctgaca tttgggcttt tggggttttg atgtgggaaa tttactccct ggggaagatg
1921 ccatatgaga gatttactaa cagtgagact gctgaacaca ttgcccaagg cctacgtctc
1981 tacaggcctc atctggcttc agagaaggta tataccatca tgtacagttg ttggcatgag
2041 aaagcagatg agcgtcccac tttcaaaatt cttctgagca atattctaga tgtcatggat
2101 gaagaatcct gagctcgcca ataagcttct tggttctact tctcttctcc acaagcccca
2161 atttcacttt ctcagaggaa atcccaagct taggagccct ggagcctttg tgctcccact
2221 caatacaaaa aggcccctct ctacatctgg ggatgcacct cttctttgat tccctgggat
2281 agtggcttct gagcaaaggc caaaaaatta ttgtgcctga aatttcccga gagaattaag
2341 acagactgaa tttgcgatga aaatattttt taggagggag gatgtaaata gccgcacaaa
2401 ggggtccaac agctctttga gtaggcattt ggtagagctt gggggtgtgt gtgtgggggt
2461 ggaccgaatt tggcaagaat gaaatggtgt cataaagatg ggaggggagg gtgttttgat
2521 aaaataaatt ctagaaagct taaaaaaaaa aaaaaaaaaa
```

TABLE 2
Amino Acid Sequence of Human BTK: Genbank Accession No. CAA41728

```
    1 maavilesif lkrsqqkkkt splnfkkrlf lltvhklsyy eydfergrrg skkgsidvek
  61 itcvetvvpe knppperqip rrgeesseme qisiierfpy pfqvvydegp lyvfspteel
121 rkrwihqlkn virynsdlvq kyhpcfwidg qylccsqtak namgcqilen rngslkpgss
181 hrktkkplpp tpeedqilkk plppepaaap vstselkkvv alydympmna ndlqlrkgde
241 yfileesnlp wwrardkngq egyipsnyvt eaedsiemye wyskhmtrsq aeqllkqegk
301 eggfivrdss kagkytvsvf akstgdpqgv irhyvvcstp qsqyylaekh lfstipelin
361 yhqhnsagli srlkypvsqq nknapstagl gygsweidpk dltflkelgt gqfgvvkygk
421 wrgqydvaik mikegsmsed efieeakvmm nlsheklvql ygvctkqrpi fiiteymang
481 cllnylremr hrfqtqqlle mckdvceame yleskqflhr dlaarnclvn dqgvvkvsdf
541 glsryvidde ytssvgskfp vrwsppevlm yskfssksdi wafgvlmwei yslgkmpyer
601 ftnsetaehi aqglrlyrph lasekvytim yscwhekade rptfkillsn ildvmdees
```

TABLE 3
Nucleotide Sequence of Murine BTK: Genbank Accession No. L29788

```
      1 aatatgtctc caggtccaga gtcttcagag atcaagtccc accttccaag tcctggcatc
    61 tcacgacgtc tggggagcta cctgcattaa gtcagaactg agtacacaaa caagttccag
  121 agagaggaag ccatggctgc agtgatactg gagagcatct ttctgaagcg ctcccagcag
  181 aaaaagaaaa catcaccttt aaacttcaag aagcgcctgt ttctcttgac tgtacacaaa
  241 ctttcatact atgaatatga ctttgaacgt gggagaagag gcagtaagaa aggttcaata
  301 gatgttgaga agatcacctg tgttgaaaca gtaattcctg aaaaaaatcc cccaccagaa
  361 agacagattc cgaggagagg tgaggagtct agtgaaatgg aacagatttc aatcattgaa
  421 aggttcccgt acccattcca ggttgtatat gatgaaggac ctctctatgt tttctcccca
  481 actgaagagc tgagaaagcg ctggattcac cagctcaaaa atgtaatccg gtacaatagt
  541 gacctggtac agaaatacca tccttgcttc tggattgatg gacagtatct ctgctgctct
  601 cagacagcca agaatgctat gggctgccaa attttggaga acaggaatgg aagcttaaaa
  661 cctgggagtt ctcatcgaaa aacgaaaaag cctcttcccc ctaccccaga ggaagatcag
  721 atcttgaaaa aaccgcttcc cccggagcca acagcagcac caatctccac aaccgagctg
  781 aaaaaggtcg tggcccttta tgattacatg ccaatgaacg caaatgactt acaattgcga
  841 aagggcgagg agtattttat cctggaggag agcaacttac cgtggtggcg agcacgagat
  901 aaaaatgggc aggaaggcta catcccaagt aactatatca ctgaagctga ggactccata
  961 gagatgtatg agtggtattc caagcacatg actcgaagtc aagctgagca actgctaaag
1021 caagagggga aagaaggagg tttcattgtc agagactcca gcaaagctgg aaaatacacc
1081 gtgtctgtgt ttgctaaatc tactggggag cctcaagggg tgatccgcca ttacgttgtg
1141 tgttccacgc cacagagcca gtattacctg gctgagaaac acctcttcag caccatccct
1201 gagctcatta actaccatca acacaactct gcaggcctca tatccaggct gaaatatcct
1261 gtgtctaaac aaaacaaaaa cgcgccttct actgcaggcc tgggctatgg atcatgggaa
1321 attgatccaa aggacctcac cttcttgaag gagcttggga ctggacaatt cggtgtcgtg
1381 aaatatggga agtggagggg ccaatatgat gtggccatca agatgatcag agaaggttcc
1441 atgtcggagg atgaattcat tgaagaagcc aaagtcatga tgaatctttc ccatgagaag
1501 ctggtgcagt tgtatggcgt ctgcaccaaa caacgcccca tcttcatcat caccgagtac
1561 atggctaatg gctgcctctt gaactacctg agggagatgc ggcaccgctt ccagacacag
1621 cagctgcttg agatgtgcaa agatgtctgt gaagcaatgg aatacttgga gtcgaagcag
1681 ttccttcaca gagacctggc agctcgaaac tgtttggtaa acgatcaagg agttgtgaaa
1741 gtatctgact ttggcctgtc taggtatgtc cttgatgatg agtacaccag ctctgtaggc
1801 tccaagtttc cagtccggtg gtctccacca gaagtgctta tgtatagcaa gttcagcagc
1861 aaatctgaca tctgggcttt tggggtttta atgtgggaga tctactccct ggggaagatg
1921 ccgtatgaga gatttactaa cagtgagaca gcagaacaca ttgctcaagg cttacgtctc
1981 tacaggcctc atctggcatc agagagggta tataccatca tgtacagctg ctggcacgag
2041 aaagcagatg aacgtcctag tttcaaaatt ctcttgagta acattctaga tgtgatggat
2101 gaagaatcct gagctggctg ctaagctccg tggatctcct cctctctcct acaaaaccta
2161 attccatgtt tcctgaggag ttccctggct gcagctctag cttccatgcg cctactgaat
2221 gcatgaagag ccctggacat ctaggaatgc ctttcttctc tcgttccctg cgatctgctc
2281 taagcaaagg tcaagggatt tctgtgccta gtattaccca taacttcaag actcctaaca
2341 gactgaattg gggacgggaa cactttgggg gagggaaaac tgtaaatagc tccactagtt
2401 gtccaacact tgttggttaa gtgttaagag tggtggtggt ggtggggggg taggaatgtt
2461 gccattaa
```

TABLE 4
Amino Acid Sequence of Murine BTK: Genbank Accession No. AAA66943

```
    1 maavilesif lkrsqqkkkt splnfkkrlf lltvhklsyy eydfergrrg skkgsidvek
  61 itcvetvipe knppperqip rrgeesseme qisiierfpy pfqvvydegp lyvfspteel
121 rkrwihqlkn virynsdlvq kyhpcfwidg qylccsqtak namgcqilen rngslkpgss
181 hrktkkplpp tpeedqilkk plppeptaap isttelkkvv alydympmna ndlqlrkgee
241 yfileesnlp wwrardkngq egyipsnyit eaedsiemye wyskhmtrsq aeqllkqegk
301 eggfivrdss kagkytvsvf akstgepqgv irhyvvcstp qsqyylaekh lfstipelin
361 yhqhnsagli srlkypvskq nknapstagl gygsweidpk dltflkelgt gqfgvvkygk
421 wrgqydvaik miregsmsed efieeakvmm nlsheklvql ygvctkqrpi fiiteymang
481 cllnylremr hrfqtqqlle mckdvceame yleskqflhr dlaarnclvn dqgvvkvsdf
541 glsryvldde ytssvgskfp vrwsppevlm yskfssksdi wafgvlmwei yslgkmpyer
601 ftnsetaehi aqglrlyrph laservytim yscwhekade rpsfkillsn ildvmdees
```

Further, derivatives and homologues of BTK may be used in the present invention. For example, nucleic acid sequences encoding BTK of the present invention may be altered by substitutions, additions, or deletions that provide for functionally equivalent-conservative variants of BTK. For example, one or more amino acid residues within the sequence can be substituted by another amino acid of similar properties, such as, for example, positively charged amino acids (arginine, lysine, and histidine); negatively charged amino acids (aspartate and glutamate); polar neutral amino acids; and non-polar amino acids.

Other conservative amino acid substitutions can be taken from the Table 5, below.

TABLE 5
Conservative amino acid replacements

| For Amino Acid | Code | Replace with any of: |
|---|---|---|
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, β-Ala, Acp |
| Isoleucine | I | D-Ile, Val, D-Val, Leu, D-Leu, Met, D-Met |
| Leucine | L | D-Leu, Val, D-Val, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, |
| | | D-Trp, Trans-3, 4, or 5-phenylproline, cis-3, 4, |
| | | or 5-phenylproline |
| Proline | P | D-Pro, L-1-thioazolidine-4-carboxylic acid, |
| | | D-or L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, D-Met, |
| | | Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Other analogs within the invention are those with modifications which increase protein stability; such analogs may contain, for example, one or more non-peptide bonds (which replace the peptide bonds) in the protein sequence. Also included are analogs that include residues other than naturally occurring L-amino acids, e.g., D-amino acids or non-naturally occurring or synthetic amino acids, e.g., β or γ amino acids.

BTK as used in the present invention may be modified by, for example, phosphorylation, sulfation, acylation, or other protein modifications. It may also be modified with a label capable of providing a detectable signal, either directly or indirectly, including, but not limited to, radioisotopes and fluorescent compounds.

It will be apparent to one of skill in the art that conventional screening assays may be used in methods of the present invention for the identification of BTK modulators. By way of example only, one BTK assay suitable for use in the present invention is a BTK Kinase assay set forth hereinbelow under “Materials and Methods”. Briefly, this assay may be used to screen for potential BTK inhibitory compounds. The effectiveness of such compounds to inhibit BTK activity may be determined based on decreased SLP 76 phosphorylation. Such compounds may then also be tested for their ability to affect bone resorption in vitro.

Further, modulators found to affect BTK activity may further be introduced into a murine osteoporosis model, such as one which has been ovariectomized (which results in a situation similar to postemopausal osteoporosis), in order to study the ability of such modulators in vivo. By way of example only, other murine model systems useful in the present invention for studying bone mass include those described in Matsushita, M., et al., Am. J. Pathol., 125:276-283 (1986) and Kuro-o M., et. el., Nature, 390:45-51 (1997).

In the present invention, techniques for screening large gene libraries may include cloning the gene library into replicable expression vectors, transforming appropriate cells with the resulting library of vectors, and expressing the genes under conditions for detection of a desired activity, e.g., binding of a ligand to BTK in the present invention. Techniques known in the art are amenable to high throughput analysis for screening large numbers of sequences created, e.g., by random mutagenesis techniques. High throughput assays can be followed by secondary screens in order to identify further biological activities which will, e.g., allow one skilled in the art to differentiate agonists from antagonists. The type of a secondary screen used will depend on the desired activity that needs to be tested.

Drug screening assays are also provided in the present invention. By producing purified and recombinant BTK of the present invention, or fragments thereof, one skilled in the art can use these to screen for drugs which are either agonists or antagonists of the normal cellular function or their role in cellular signaling. In one aspect, the assay evaluates the ability of a compound to modulate binding between BTK of the present invention and a naturally occurring ligand. The term “modulating” encompasses enhancement, diminishment, activation or inactivation of BTK activity. Assays useful to identify ligands to BTK of the present invention, including peptides, proteins, small molecules, and antibodies, that are capable of binding to BTK and modulating its activity, are enocompassed herein. A variety of assay formats may be used in the present invention and are known by those skilled in the art. One example of a BTK inhibitor is LFM-A13 (Mahajan S., et al., J. Biol. Chem. 274(14):9587-99 (1999).

In many drug screening programs which test libraries of compounds and natural extracts, high throughput assays are desirable in order to maximize the number of compounds surveyed in a given period of time. Assays which are performed in cell-free systems, such as may be derived with purified or semi-purified proteins, are often preferred as primary screens in that they can be generated to permit rapid development and relatively easy detection of an alteration in a molecular target which is mediated by a test compound.

Compounds identified using assays, as discussed hereinabove, may be antagonists or agonists of BTK, and may bind to BTK, thereby modulating BTK activity. The term “modulating” encompasses enhancement, diminishment, activation or inactivation of BTK activity. Ligands to BTK of the present invention, including peptides, proteins, small molecules, and antibodies, that are capable of binding to BTK and modulating its activity, are encompasses herein. These compounds are useful in modulating the activity of BTK and in treating BTK-associated disorders.

“BTK-associated disorders” refers to any disorder or disease state in which the BTK protein plays a regulatory role in the metabolic pathway of that disorder or disease. Such disorders or diseases include, but are not limited to, osteoporosis. As used herein the term “treating” refers to the alleviation of symptoms of a particular disorder in a patient, the improvement of an ascertainable measurement associated with a particular disorder, or the prevention of a particular immune, inflammatory or cellular response.

A compound which acts as a BTK modulator may be administered for therapeutic use as a raw chemical or may be the active ingredient in a pharmaceutical formulation. Such formulations of the present invention may contain other therapeutic agents as described below, and may be formulated, for example, by employing conventional solid or liquid vehicles or diluents, as well as pharmaceutical additives of a type appropriate to the mode of desired administration (for example, excipients, binders, preservatives, stabilizers, flavors, etc.) according to techniques such as those well known in the art of pharmaceutical formulation.

Compounds of the present invention may be administered by any suitable means, for example, orally, such as in the form of tablets, capsules, granules or powders; sublingually; buccally; parenterally, such as by subcutaneous, intravenous, intramuscular, or intrasternal injection or infusion techniques (e.g., as sterile injectable aqueous or non-aqueous solutions or suspensions); nasally such as by inhalation spray; topically, such as in the form of a cream or ointment; or rectally such as in the form of suppositories; in dosage unit formulations containing non-toxic, pharmaceutically acceptable vehicles or diluents.

Such compounds may, for example, be administered in a form suitable for immediate release or extended release. Immediate release or extended release may be achieved by the use of suitable pharmaceutical compositions comprising compounds of the present invention, or, particularly in the case of extended release, by the use of devices such as subcutaneous implants or osmotic pumps. Compounds of the present invention may also be administered liposomally.

Exemplary compositions for oral administration include suspensions which may contain, for example, microcrystalline cellulose for imparting bulk, alginic acid or sodium alginate as a suspending agent, methylcellulose as a viscosity enhancer, and sweeteners or flavoring agents such as those known in the art; and immediate release tablets which may contain, for example, microcrystalline cellulose, dicalcium phosphate, starch, magnesium stearate and/or lactose and/or other excipients, binders, extenders, disintegrants, diluents and lubricants such as those known in the art.

Compounds of the present invention may also be delivered through the oral cavity by sublingual and/or buccal administration. Molded tablets, compressed tablets or freeze-dried tablets are exemplary forms which may be used. Exemplary compositions include those formulating the compound(s) of the present invention with fast dissolving diluents such as mannitol, lactose, sucrose and/or cyclodextrins.

Also included in such formulations may be high molecular weight excipients such as celluloses (avicel) or polyethylene glycols (PEG). Such formulations may also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g., Carbopol 934). Lubricants, glidants, flavors, coloring agents and stabilizers may also be added for ease of fabrication and use.

Exemplary compositions for nasal aerosol or inhalation administration include solutions in saline which may contain, for example, benzyl alcohol or other suitable preservatives, absorption promoters to enhance bioavailability, and/or other solubilizing or dispersing agents such as those known in the art.

Exemplary compositions for parenteral administration include injectable solutions or suspensions which may contain, for example, suitable non-toxic, parenterally acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid.

Exemplary compositions for rectal administration include suppositories which may contain, for example, a suitable non-irritating excipient, such as cocoa butter, synthetic glyceride esters or polyethylene glycols, which are solid at ordinary temperatures, but liquify and/or dissolve in the rectal cavity to release the drug.

Exemplary compositions for topical administration include a topical carrier such as Plastibase (mineral oil gelled with polyethylene).

The effective amount of a compound of the present invention may be determined by one of ordinary skill in the art, and includes exemplary dosage amounts for an adult human of from about 0.1 to 100 mg/kg of body weight of active compound per day, which may be administered in a single dose or in the form of individual divided doses, such as from 1 to 4 times per day. It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats and the like, subject to BTK-associated disorders.

The compounds of the present invention may be employed alone or in combination with each other and/or other suitable therapeutic agents useful in the treatment of BTK-associated disorders.

In another aspect, the present invention relates to the use of an isolated nucleic acid in “antisense” therapy. As used herein, “antisense” therapy refers to administration or in situ generation of oligonucleotides or their derivatives which specifically hybridize under cellular conditions with the cellular MRNA and/or genomic DNA encoding BTK of the present invention so as to inhibit expression of the encoded protein, e.g., by inhibiting transcription and/or translation. In general, “antisense” therapy refers to the range of techniques generally employed in the art, and includes any therapy which relies on specific binding to oligonucleotide sequences.

Gene constructs useful in antisense therapy may be administered may be administered in any biologically effective carrier, e.g., any formulation or composition capable of effectively delivering a nucleic acid sequence to cells in vivo. Approaches include insertion of the subject gene in viral vectors including recombinant retroviruses, adenoviruses, adeno-associated viruses, and herpes simplex virus-1, or recombinant bacterial or eukaryotic plasmids. Viral vectors transfect cells directly; an advantage of infection of cells with a viral vector is that a large proportion of the targeted cells can receive the nucleic acid. Several viral delivery systems are known in the art and can be utilized by one practicing the present invention.

In addition to viral transfer methods, non-viral methods may also be employed. Most non-viral methods of gene transfer rely on normal mechanisms used by mammalian cells for the uptake and intracellular transport of macromolecules. Exemplary gene delivery systems of this type include liposomal derived systems, poly-lysine conjugates, and artificial viral envelopes. Nucleic acid sequences may also be introduced to cell(s) by direct injection of the gene construct or by electroporation.

In clinical settings, the gene delivery systems can be introduced into a patient by any of a number of methods, each of which is known in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof.

The pharmaceutical preparation of the gene therapy construct can consist essentially of the gene delivery system in an acceptable diluent, or can comprise a slow release matrix in which the gene delivery vehicle is embedded. Alternatively, where the complete gene delivery system can be produced intact from recombinant cells, e.g., retroviral vectors, the pharmaceutical preparation can comprise one or more cells which produce the gene delivery system.

Proteomic analysis of RANKL-induced signal transduction intermediates from RAW 264 cells (murine macrophage cell line) was conducted as set forth in the Examples below. From this analysis, it can be seen that RANKL induces specific tyrosine phosphorylation of BTK, establishing the importance of BTK in the process of RANKL-induced osteoclast activation. As such, BTK is an important target in the treatment and prevention of osteoporosis.

The following section sets forth materials and methods used in the present invention, and which were utilized in the Examples set forth hereinbelow.

### MATERIALS AND METHODS

BTK^{-/-} mice: Described in the literature. (Khan W. N., et al., Immunity 3(3):283-299). The BTK^{-/-} mice have a mixed genetic background of 129/Sv×C57BL/6. For wild-type controls, 129/Sv×C57BL/6 or C 57BL/6 mice were used.

BTK^{xid} mice and BTK^{1o} mice: The BTK transgenic constructs have been described in the literature. (Satterthwaite A. B., et al., Proc. Natl. Acad. Sci. U S A. 94(24):13152-13157; Satterthwaite A. B., et al., Proc. Natl. Acad. Sci. USA. 97(12):6687-6692). The BTK^{xid} and BTK^{1o} mice in the present invention are transgenic Ba1b/C mice derived from the BTK transgenic constructs which contain either the xid phenotype or one or two copies of the murine BTK cDNA transgene driven by the Ig heavy chain promoter and enhancer on a BTK xid background. The transgene expresses approximately 25% of endogenous BTK protein levels in splenic B cells.

Bone Scan: The total and trabecular density of the proximal tibia were evaluated in ex-vivo mouse bone samples using an XCT Research SA pQCT (Stratec Medizintechnik, Pforzheim, Germany). The bone was placed in a sample holding tube, and positioned within the gantry of the instrument so that the tibia was in the scanning field. A two-dimensional scout scan was run for a length of 10 mm. After the scout view was displayed on the monitor, the pQCT scan was initiated 1.4 mm distal to the epiphysis. The scan was 1 mm thick, had a voxel (three-dimensional pixel) size of 90 μm, and consisted of 180 projections. After the scan was completed, the cross-sectional image was displayed on the monitor. A region of interest was outlined around the tibia. Using an iterative algorithm, soft tissue (density below 223 mg/cm³) was automatically removed. The density of the remaining bone was reported as total density (mg/cm³). The outer 55% of the bone was peeled away in a concentric fashion to determine trabecular density (mg/cm³).

Generation of recombinant BTK constructs: Primers based on published sequence data (sense: 5′-atacggatccgccgccaccatggctgcagtgatactg-3′ (SEQ ID NO:5), antisense: 5′ -tgacgcggccgctcaggattcttcatccatc-3′ (SEQ ID NO:6) (Sigma Genosys)) were used to amplify full length murine BTK from Marathon Ready mouse spleen cDNA (Clontech) with Advantaq polymerase (Clontech). Cycling conditions in a Perkin Elmer 9600 thermocycler were as follows: Initial denaturation of 94° C. for 2′ (minutes), 5 cycles of 94° C. for 30′, 50° C. for 30′, 68° C. for 2′, 5 cycles of 94° C. for 30′, 55° C. for 30′, 68° C. for 2′, 30 cycles of 94° C. for 30′, 65° C. for 30′, 68° C. for 2′, then 68° C. for a 7′ extension. The resulting 2 kb fragment was isolated from a 1% agarose gel via Quantum Prep Freeze and Squeeze gel purification (BioRad), cloned into PCR2.1TOPO (Invitrogen), electroporated into TOP10 cells (Invitrogen) and spread on LB plates containing 100 ug/mL ampicillin and X-gal. Individual 5 ml cultures of LB containing 100 ug/mL ampicillin were inoculated with white colonies and grown overnight at 37° C. with shaking. DNA was obtained (Qiagen robot) and positive clones were selected by restriction enzyme analysis, which was confirmed by sequence analysis. BamHI digested mBTK was cloned into BamHI digested/CIAP treated p3XFLAG-CMV10 expression vector (Sigma). NotI/KpnI digested insert was cloned into NotI/KpnI digested pCDNA 3.1-(Invitrogen).

Generation of recombinant BTK mutants: The Quickchange Site-directed mutagenesis kit (Stratagene) was used to make nucleotide mutations resulting in amino acid changes Arg-28-Cys (XID), Glu-41-Lys (Gain of Function), and Lys-430-Arg (Dominant Negative) in mBTK. Complementary oligonucleotides to the following regions were synthesized and PAGE purified (Sigma-Genosys): R28C: sense-5′-cctttaaacttcaagaagtgcctgtttctcttgactg-3′ (SEQ ID NO:7), complementary antisense-5′-cagtcaagagaaacaggcacttcttgaagtttaaagg-3′ (SEQ ID NO:8) (mBTK nucleotides 57-94), E41K: sense-5′-ctttcatactataaatatgactttgaacgtggg-3′ (SEQ ID NO:9), complementary antisense-5′-cccacgttcaaagtcatatttatagtatgaaag-3′ (SEQ ID NO: 10) (mBTK nucleotides 102-134), K430R: sense-5′-ccaatatgatgtggccatcagaatgatcagagaaggttc-3′ (SEQ ID NO: 11), complementary antisense-5′-gaaccttctctgatcattctgatggccacatcatattg-3′ (SEQ ID NO: 12) (mBTK nucleotides 1262-1300).

The oligonucleotide set corresponding to each mutation was annealed to full length mBTK in pcDNA3.1- with Quickchange kit components and cycled in a Perkin Elmer 9600 thermocycler as follows: Initial denaturation of 95° C. for 30′, 15 cycles of 95° C. for 30′, 55° C. for 1′, 68° C. for 16′, then 68° C. for a 1′ extension. The methylated parental DNA strand was eliminated by digesting the entire reaction with DpnI for 60′ at 37° C. 1 uL was transformed into XL-1 Blue competent cells and plated onto LB plates containing 100 ug/mL ampicillin. Individual 5 ml cultures of LB containing 100 ug/mL ampicillin were inoculated with colonies and grown overnight at 37° C. with shaking. DNA was obtained (Qiagen robot) and mutations verified by sequence analysis. N-terminal FLAG constructs of all mutants were generated by inserting BamHI digested fragments into BamHI digested and CLAP treated p3XFLAG-CMV10 expression vector.

Generation of mBTK pooled stable cell lines: 0E6 RAW 264.7 precursor cells were seeded onto 100 mm dishes 18 hours prior to transfection. Lipofectamine Plus (Invtrogen/Gibco-BRL) was used for transfection. 4 ug Qiagen maxi prep derived DNA of all untagged and FLAG-tagged wild-type and mutant mBTK in both pcDNA3.1- and p3XFLAG-CMV 10 were separately combined with 20 uL Lipofectamine Plus and 500 uL Optimem, incubated at room temperature (RT) for 15′ and then combined with a mixture of 500 uL Optimem and 30 uL Lipofectamine for 15′ RT. The mixture was drizzled in a dropwise manner onto the plates in which growth media had been replaced with 5 mL Optimem media. Plates were incubated for 3 hours 37° C./5% CO₂ at which time Optimem was removed and replaced with growth medium. 24 hours post-transfection, media was replaced with that containing 900 ug/mL G418.

Cell culture: RAW 264 cells were obtained from Bristol-Myers Squibb Pharmaceutical Research Institute, Department of Metabolic Diseases, and prepared as follows: Cells were grown in minimal essential media supplemented with 5% fetal bovine serum and 1% nonessential amino acids. For assay purposes, RAW 264 cells were starved for 5 hours in serum free media and then cultured in media containing 2% fetal bovine serum and RANK ligand. When inhibitors were used, the cells were pre-exposed to the inhibitor for one hour prior to RANKL stimulation.

Western Blot Analysis of FLAG-BTK Mutants: Confluent RAW 264.7 cells expressing either FLAG vector alone, FLAG-BTK wild-type, FLAG-BTK R28C, FLAG-BTK E41K or FLAG-BTK K430R were washed twice with ice cold PBS and lysed on ice in FLAG-IP lysis buffer (50 mM Tris-HCl [pH=7.4], 150 mM NaCl, 1 mM EDTA, 1% Triton X-100, 1 mM sodium orthovanadate, 1×Boehringer protease inhibitor, 1×Sigma phosphatase inhibitor). Lysates were scraped, Dounce homegenized 50 strokes with a tight pestle, transferred to 1.5 ml microfuge tubes, microfuged at 14,000 rpm for 15′ and supernatant collected and stored at −80° C. α-FLAG immunoprecipitations were performed on equivalent amounts of lysate in 1 ml total FLAG-IP lysis buffer containing 20 μl α-FLAG Protein-A Agarose (Sigma). Immunoprecipitations were done with rocking for 2 hours at 4° C., pelleted and washed 4× with TBS. Samples were boiled 3′ in Laemmli buffer containing DTT, run on 10% acrylamide Bis-Tris gels (Novex) for 50′ at 200 V using MOPS running buffer and blotted onto PVDF for 1 hour at 30 V. Blots were blocked in TBST (TBS+0.05% Tween) containing 1% BSA for 1 hour at room temperature.

Blots were then probed with either α-FLAG-HRP (1:500, UBI) or α-Phosphotyrosine-4G10 (1:2000, UBI) in TBST-BSA for 1 hour at room temperature. Blots were then washed 4 times, 5 minutes each, in TBST and either reacted with Amersham ECL+Plus chemiluminescence kit (α-FLAG) or probed with a secondary antibody (α-phosphotyrosine blot probed with α-mouse IgG-HRP, 1:30,000, 1 hr, washed 4 times in TBST) and then reacted with ECL+Plus. Bands were visualized using a Fluor-S MAX (Bio-Rad) and quantitations done using Quantity One image analysis software (Bio-Rad).

IP Kinase Assays: FLAG-BTK wild-type and mutant proteins were immunoprecipitated as described hereinabove under “Western Blot Analysis of FLAG-BTK Mutants.” Immunoprecipitates were washed 3 times with TBS and once with BTK kinase buffer (138 mM NaCl, 50 mM Tris [pH=8.0], 10 mM MgCl2, 10 mM MnCl2). Immunoprecipitates were then resuspended in BTK kinase buffer containing 50 nanograms of purified recombinant SLP-76 as substrate and 40 μM ATP. Control reactions contained recombinant BTK alone, SLP-76 alone or mock immunoprecipitation (no lysate) with SLP-76. Kinase reactions were carried out for 5′ at 37° C., microfuged briefly, supernatants placed on ice, Laemmli buffer added and reactions boiled 3′. Samples were then run on a 10% acrylamide gel, blotted, probed with α-phosphotyrosine, visualized and quantitated exactly as described hereinabove under “Western Blot Analysis of FLAG-BTK Mutants.”

BTK Kinase Assay and ELISA Protocol

Materials

Sodium Carbonate buffer 0.05 N. pH 8 (Sigma C-3041) for coating the substrate

Immulon 2 Elisa Plates (Dynatech 0110103455)

PBS 1× pH 7.5

Washing buffer (Tween 20 @ 0.05% final in PBS 1×)

Blocking buffer [Sanofi Diagnostics blocking buffer 1×(#0220-96)]

Chromogen mixture=50% Kirkegaard & Perry labs #50-76-01 TMB

50% Kirkegaard & Perry Labs #50-65-00 Peroxidase

Protocol

1. Coat Plates with the substrate (GST-LAT full length protein 50 ng/well in 100 μl of Sodium Carbonate buffer. Incubate ON @ 4° C.

2. Wash plates with PBS Tween 20

3. Block Plates with blocking buffer (100 μl/well). Incubate 90 min @ RT.

4. Wash plates with PBS, Flick plate dry

5. Add 9 ng/well recombinant GST-BTK-KD (Kinase Domain) 100 μl/well in

kinase buffer (25 mM Heaps pH 7.5, 5 mM MgCl₂, 5 mM MnCl₂, 0.1% BSA, 10 μM ATP).

6. Incubate 60 min 37° C.)

7. Wash plates with PBS Tween 20

8. Add anti-phosphotyrosine mAb 100 μl 1/1000 final dilution (PY99-HRP, Santa Cruz Biotechnology #7020) in blocking buffer (45 min @RT)

9. Wash as above

10. Add 100 μl/well of Chromogen mixture (Incubate apron. 3-5 min RT) Quench w/ 100 ul 0.1N Sulfuric Acid. Read @ 450/570 nm

Total Lysate α-Phosphotyrosine Western Blot: Cells were lysed and 20 μg of total lysate from each sample was electrophoreses, blotted, probed with α-phosphotyrosine and visualized as described hereinabove under “Western Blot Analysis of FLAG-BTK Mutants.”

Immunofluroescence microscopy: FLAG-tagged mBTK wild-type and stable cell line pools were separately seeded at a density of 10E6 cells per 100 mm dish each containing collagen I coated oversleeps (Becton Dickinson). Media was replaced after six hours with media containing 200 ng/mL RANK-ligand. On day 5 post-stimulation, media was removed, replaced with 5 mL ice-cold 4% paraformaldehyde/0.1 % Triton-X, and the plates incubated at 4° C. for 30′. Plates were washed 3×5 mL ice-cold 1×DPBS/0.1% Triton-X then blocked with 5 mL 1×DPBS/0.1% Triton-X containing 4% non-fat dry milk at 4° C. for 60′. Blocking buffer was replaced with 2 mL rhodamine-phalloidin (Molecular Probes), diluted 1:40 in DPBS/0.1% Triton-X containing 4% non-fat dry milk, and incubated at 4° C. for 10′. Plates were washed 3×5 mL ice-cold 1×DPBS/0.1% Triton-X. Oversleeps were each removed and mounted cell slide down, using prolong Antipode mounting media (Molecular Probes), onto glass slides and dried overnight. Rhodamine-phalloidin bound actin was visualized with 530 nmDF35 excitation/580DF30 emission filters in a Zeus Axioscop 2 microscope. Images were captured with Optronics DEI-750 Acquire software.

Subcellular fractionation: RAW 264.7 cells were washed twice with ice-cold PBS containing 1 mM sodium orthovanadate and lysed for 5 minutes inTriton X-100 lysis buffer (10 mM Tris pH 7.4, 1 mM EDTA, 0.5% Triton X-100, 1 mM sodium orthovanadate, 1 mM NaF, 10 μg/m leupeptin, 1 TIU/ml aprotinin, and 1 mM PMSF on ice. This aliquot represented the cytosolic fraction. For cytoskeletal proteins, remaining cells were lysed in RIPA buffer (150 mM NaCl, 10 mM Tris-HCl pH 7.4, 1 mM EDTA, 1% Triton X-100, 1% deoxycholate, 0.1% SDS, 1 mM sodium orthovanadate, 1 mM NaF, 10 μg/ml leupeptin, 1 TIU/ml aprotinin, and 1 mM PMSF) for 5 minutes on ice. The cytoskeletal proteins were separated by centrifugation at 16,000×gravity at 4° C. for 15 minutes.

Electrophoresis: Isoelectric focusing was carried out in Pharmacia IPG strips, pH 3-10 nonlinear gradient for approximately 150,000 Vhr. Following equilibration for 15 min in 10% glycerol, 50 mM DTT, 2.3% SDS, and 62.5 mM Tris pH 6.5), the IPG strip was layered onto the top of a 10% acrylamide slab gel (1.00 mm thick), and SDS slab gel electrophoresis was carried out for 5 hours at 20 watts/gel. The slab gels were transferred overnight to PVDF membrane which were then were fixed in a solution of 10% acetic acid-40% methanol for 30 min. followed by staining overnight with the fluorescent dye Sypro Ruby Red (Molecular Probes, Eugene, Oreg.) as described in the manufacturer's protocol. Maximal fluorescence incorporation occured within 4 hours. For Western blots, the PVDF membranes were blocked for >2 hours with 1% bovine serum albumin (BSA) (w/v) in 1% Tween-Tris buffered saline (TTBS) (v/v), rinsed in TTBS, incubated with primary antibody diluted 1:2,500 in 1% BSA-TTBS for 2 hours, rinsed in TTBS, and incubated with secondary antibody diluted 1:5,000 in TTBS for 1 hour. The blot was rinsed with TTBS, and treated with ECL (Pharmacia-Amersham Biotech, Piscatawy, N.J.). Images were generated using a BioRad Fluor-S Max imaging system. The images were then interpreted using PDQuest 6.1 software (BioRad Laboratories Hercules, Calif.). Samples were selected for in-gel digestion based upon information obtained from digital images generated from chemilumenescent stained western blots compared to Sypro fluorescent stained gel images.

Analytical Biochemistry and Mass Spectrometry

In-gel Digestion: Selected protein spots from Sypro stained membranes were excised and washed twice with water for 15 min. Samples were then dried under vacuum in a Savant SpeedVac. The samples were then reduced and alkylated and the gel pieces were washed with 50% acetonitrile:100 mM ammonium bicarbonate (v/v) and dried again under vacuum. The gel pieces were then rehydrated with ammonium bicarbonate containing 12.5 ng trypsin and incubated overnight at 37° C. Following digestion, the gel pieces were extracted with 50% acetonitrile: 100 mM ammonium bicarbonate (v/v) and the supernatants dried under vacuum. The dried material was resuspended in formic acid for mass spectral analysis.

Liquid chromatography-mass spectrometry: Following the extraction of peptides from the gel pieces, the samples were evaporated to dryness (Model AES2010, Savant Instruments, Holbrook, N.Y.). The peptides were dissolved in 5% formic acid, vortexed, sonicated, and then briefly centrifuged to settle insoluble matter. The samples were then loaded onto capillaries packed with a stirred slurry of POROS R2/H (PE-Biosystems, Framingham, Mass.) using an argon pressure reservoir. The capillaries were pre-equilibrated with >10 column volumes of mobile phase A (A=0.2% isopropanol, 0.1% acetic acid, 0.001% trifluoroacetic acid) prior to the sample loading process. The chromatographic separation was preformed with a gradient of increasing organic concentration of 0% B-100% B (B=A+80% acetonitrile) in 45 min at an initial applied pressure of 22 bar generated using a binary HPLC pump (Model 1100, Hewlett-Packard, Palo Alto, Calif.) flowing at 250 microliters per min. prior to the split. The applied electrospray voltage was 2.2 kV. No sheath gases or make up flows were applied, although the mass spectrometer's heated capillary was operated at 150° C. The sample was sprayed into a Model TSQ7000, (Finnigan, San Jose, Calif.). The third quadrupole of the mass spectrometer was scanned over the mass to charge range of 475 to 1800 in 1.0 sec. If ions present in this mass range exceeded 80,000 counts, then the three most intense ions present in the spectra were subjected to collision induced dissociation. The collision cell was operated at ˜3 mtorr, while the applied collision voltage was adjusted for each precursor ion by multiplying each ion's mass to charge ratio by a factor of 26. The scanned range for the MS/MS scans were also mass to charge dependent, scanning up to a ratio twice that of the precursor ion's apparent mass to charge. The mass spectral data was analyzed by SEQUEST (ver. 27PVM, Finnigan) on a supercomputer built in-house. The output files were then each viewed to verify the accuracy of the protein assignments.

As illustrated in the following Examples, it is now found in the present invention that BTK is a critical enzyme in bone resorption and, accordingly, clinical osteoporosis.

### EXAMPLE 1

### Effect of Deficiency of BTK Gene on Bone Morphology

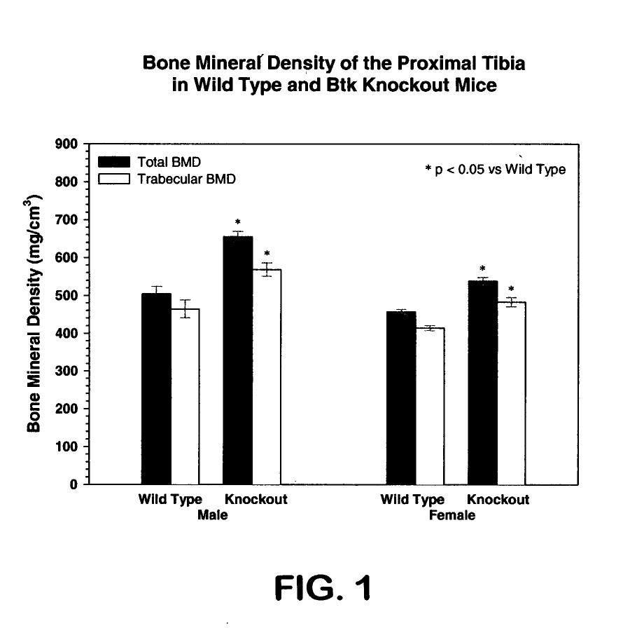

The proximal tibial bones of BTK^{-/-} (knockout) mice and their wildtype counterparts were evaluated to determine the effect of alteration of the BTK gene on bone morphology. The results of the bone mineral density analysis by tomography shown FIG. 1 indicate that tibial sections from BTK^{-/-} mice showed evidence of osteopetrosis compared to the wild-type mice. These results show that BTK is a critical enzyme in the process of bone resorption.

### EXAMPLE 2

### Effect of Mutation of BTK Gene on Bone Morphology

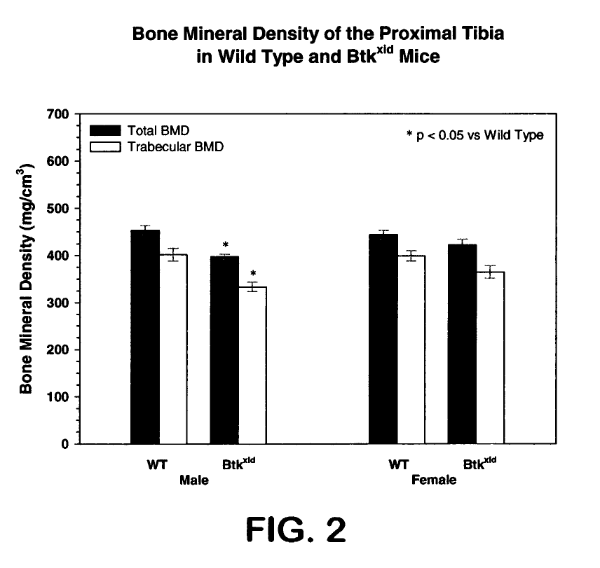

The tibial sections from BTK^{xid} mice and their wild-type counterparts were evaluated to determine the effect of the xid mutation on bone morphology. The results of the bone mineral density analysis by tomography for these mice is shown in FIG. 2 and indicate that tibial section for BTK^{xid} mice showed evidence of osteoporosis compared to the wild-type mice.

This data not only confirms BTK as a key intermediate in the bone resorption process, but also shows that a single point mutation, which would presumably render the protein inactive due to the inability of the BTK to bind to PIP₃, can in fact reverse the observed bone phenotype previously observed with the knockout mice from osteopetrotic to osteoporotic. To test this possibility, transgenic mice in which copies of the BTK transgene were added back onto the xid background were utilized to determine whether a reversal of the osteopenic phenotype was possible. The BTK transgene BTK^{lo,} has previously been reported to have an enzymatic activity approximately 25% that of the wild type BTK enzyme. (Satterthwaite A. B., et al., J. Exp. Med. 188(5):833-44 (1998)).

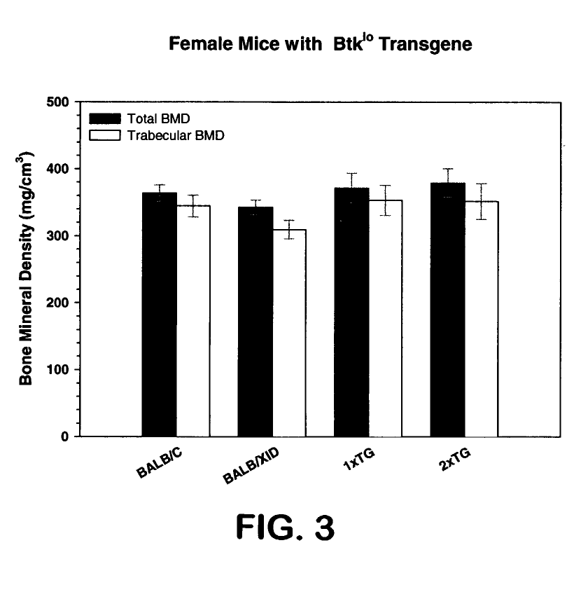

1XTG indicates one copy of the transgene that has approximately 25% wild type level of enzyme and 2X represents two copies of the transgene. Wild-type (wt) plus one or two copies of the transgene would have 100% the normal level of BTK plus the additional 25% above that for each copy of the transgene present. Therefore, it is possible to have animals with 0, 25, 50. 100, 125 and 150% or wild type levels. In FIG. 3, the results from the bone mineral density analysis indicate that tibia from BTK xid female mice in which the BTK is added back in one or two copies as a transgene, show a trend of increased bone mineral density with the addition of two copies of the normal transgene able to completely compensate for the observed osteopenic xid defect.

These results show that BTK is a critical enzyme in the process of bone resorption, and further show that a single point mutation can reverse the observed bone phenotype previously observed with the knockout mice.

### EXAMPLE 3

### BTK Molecular Constructs

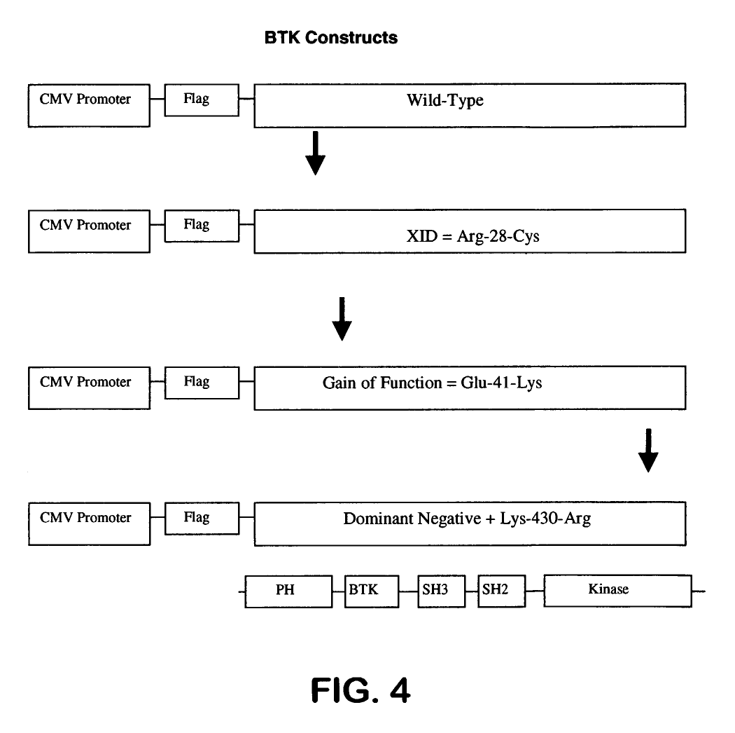

Four molecular constructs for overexpression of BTK were designed, and are summarized in FIG. 4. Mouse BTK wild-type and three point mutations were cloned into pCDNA 3.1 and p3XFLAG for molecular tagging (3 FLAG epitopes). In the initial construct, wild-type mouse BTK was placed under control of the CMV promoter with a FLAG amino sequence attached to the amino terminal end of the BTK protein coding sequence. Other constructs using the same general design were generated from this construct: (1) the xid mutation, which contains a point mutation at residue 28 converting arginine to cysteine; (2) a “gain of function” mutation which was reported in one hemapoetic cell line in which residue 41 was converted from glutamic acid to lysine; and (3) a dominant negative mutation in which the lysine at residue 430 was converted to an arginine, and which is intended to obliviate the kinase activity of the protein.

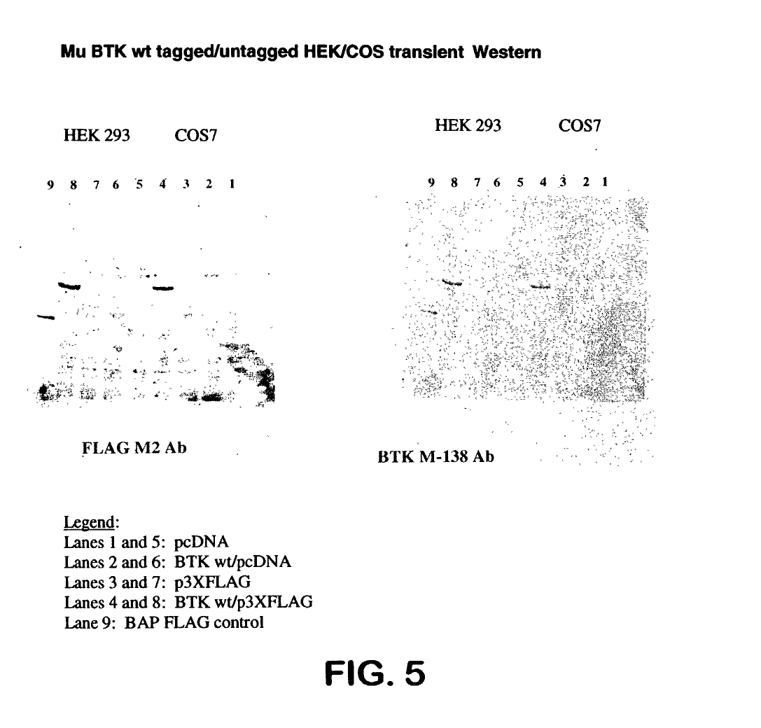

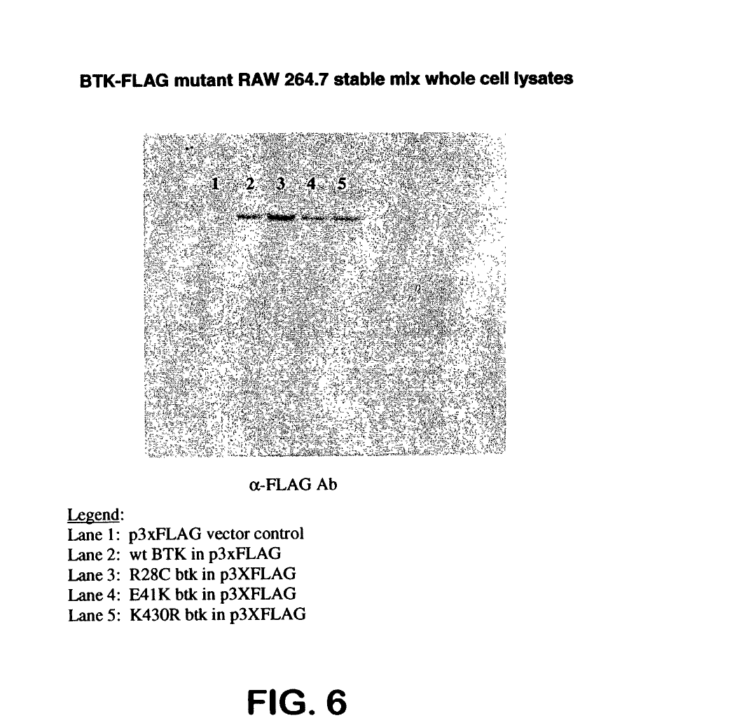

Constructs were initially transfected into HEK 293 and COS 7 cell lines. As shown in FIG. 5, the FLAG-tagged BTK constructs were successfully expressed in both cell lines. FLAG BTK was detected in transfected COS-7 and HEK 293 lysates with FLAG and BTK COOH terminal 20 amino acid antibodies (detects both FLAG and untagged). These constructs were then transfected into RAW 264.7 cells and cell lysates were examined for expression of FLAG tagged BTK. As shown in FIG. 6, transfection and expression into RAW 264.7 cells was successful. FLAG BTK was detected in transfected stable RAW 264.7 cell lysates.

This data establishes that BTK and various constructs thereof may be cloned and stably expressed in a variety of mammalian cell lines. Particularly, BTK constructs may be stably expressed in osteoclast progenitor cell lines.

### EXAMPLE 4

### BTK Assays

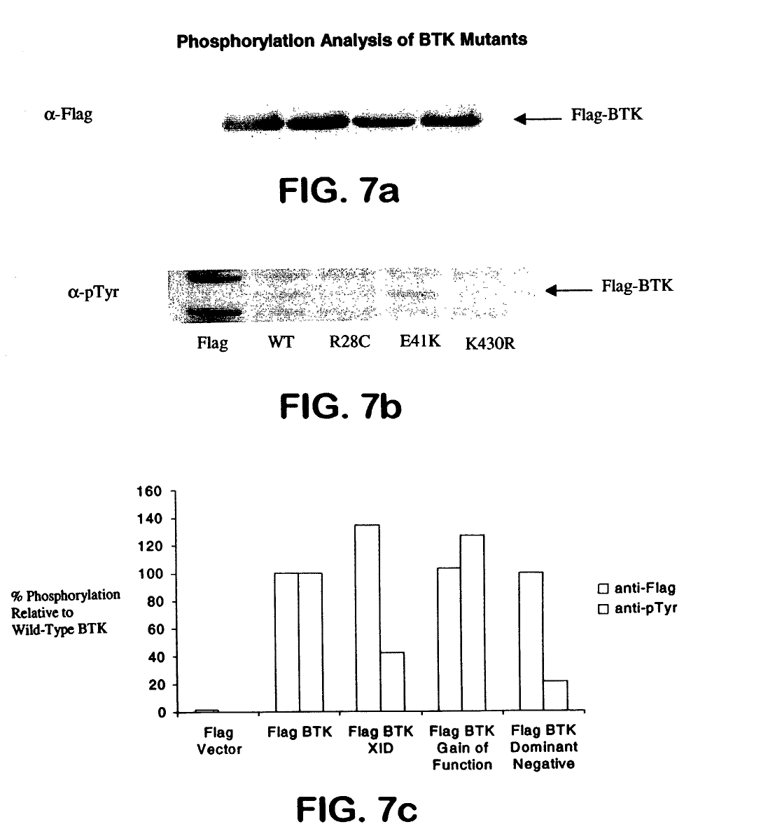

Raw 264.7 cells containing the four molecular constructs of BTK were lysed, and the BTK was immunoprecipitated with anti-FLAG antibody. The immunoprecipitated FLAG tagged BTK was Western blotted in duplicate with either anti FLAG antibody or anti-phosphotyrosine antibody and analyzed by chemiluminescence. The mean fluorescence value of the wild-type FLAG tagged BTK was normalized to 100%. As shown in FIG. 7a, FLAG BTK was detected in transfected stable RAW 264.7 cell lysates with FLAG antibody. As shown in FIG. 7b, phosphotyrosine labeled BTK was detected in the same transfected stable RAW 264.7 cell lysates. FIG. 7c shows a fluorometric densitometry analysis of antiflag fluorescence versus anti-phosphotyrosine fluorescence for FLAG tagged BTK and mutants.

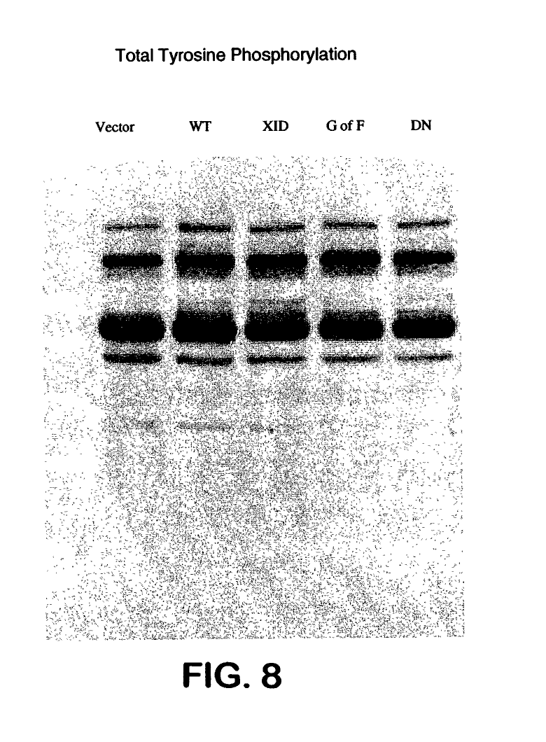

As illustrated in FIG. 7c, the xid BTK phosphorylation is reduced significantly compared to the wild-type BTK, whereas the “gain of function” construct appeared to show a slight increase in BTK tyrosine phosphorylation. The dominant negative construct showed reduced tyrosine phosphorylation. Transfected stable RAW 264.7 cell lysates were then blotted with antiphosphotyrosine to show equivalent levels of total cellular tyrosine phosphorylation, the results of which are shown in FIG. 8.

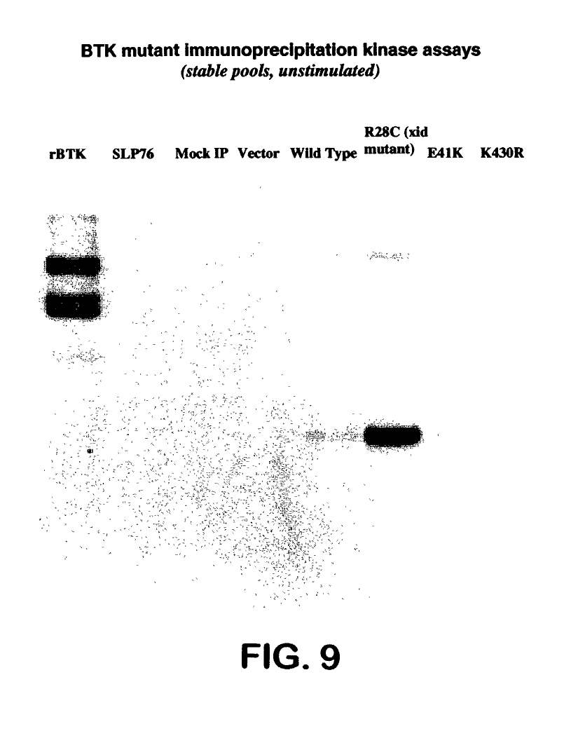

The BTK constructs were then immunoprecipitated from whole cell lysates with anti-FLAG antibody and used to phosphorylate the known BTK substrate, SLP 76, in an in vitro assay, the results of which are shown in FIG. 9. The phosphorylation intensity was monitored by incorporation of phosphotyrosine into the substrate. As shown in FIG. 9, recombinant FLAG tagged BTK is able to autophosphorylate itself in the assay, whereas SLP 76 cannot undergo autophosphorylation. In mock and vector alone immunoprecipitations, there is no phosphorylation of the SLP 76 target. However, addition of immunoprecipitated wild-type BTK resulted in phosphorylation of both BTK itself and the SLP 76 target. Addition of BTK immunoprecipitated from the xid construct cell lysate resulted in hyperphosphorylation of the SLP 76 target as well as increased phosphorylation of BTK itself. Phosphorylation using extracts from the “gain of function” and dominant negative mutant cell contracts appeared to be reduced compared to the wild-type BTK.

These results establish that the BTK^{xid} mutation represents a hyperactive form of the enzyme and offers explanation of the role for the xid mutation in the establishment of osteopenia in mice. These results further suggests that downstream effector proteins such as, but not limited to, SLP 76 may also contribute to this osteopenic effect by virtue of an increased activity as well as differential compartmentalization of the mutant BTK.

### EXAMPLE 5

### Cell Biology

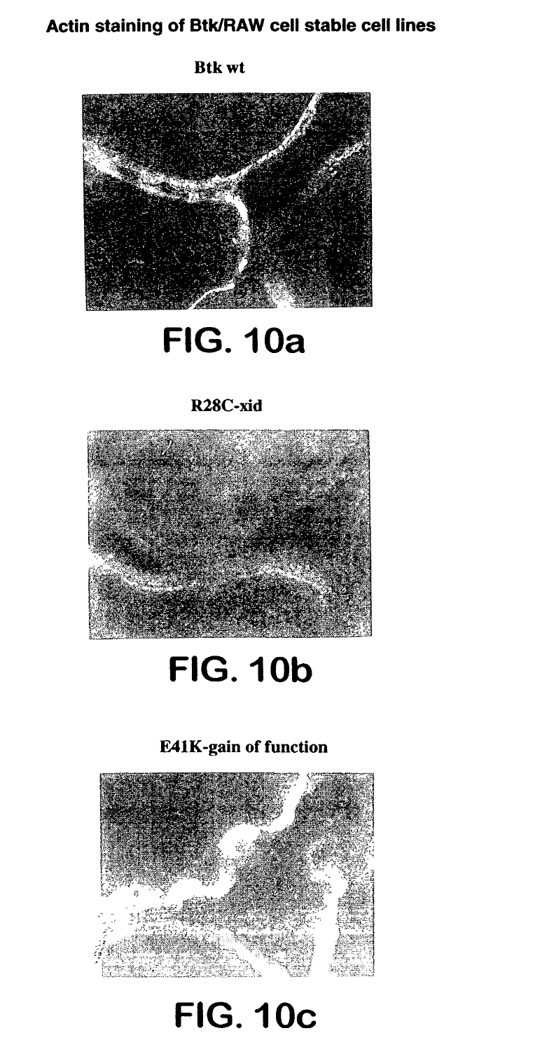

Raw 264.7 cells containing the hereinabove stated molecular constructs of BTK were stained with phalloidin and analyzed by fluorescence microscopy. Subtle differences were seen between different mutants stained with actin/phalloidin. As shown in FIG. 10a, the wild-type BTK expressing cell showed a single ring of podosomes, some stress fibers, and cytoplasmic staining. As shown in FIG. 10b, the R28C (xid) showed a double ring of podosomes. These irregularly shaped cells also possessed larger and multiple sealing zones. As shown in FIG. 10c, the E41K (“gain of function”) showed numerous large cells containing a lot of stress fibers. BTK Ab showed localization at or near the membrane regardless of mutation. This cell biology data links BTK activation to podosome assembly as well as formation of sealing zones which are necessary structures for subsequent bone resorption by activated osteoclasts.

The xid form of BTK which has shown evidence of hyperactivity in vitro as well as increased osteopenia in vivo leads to increased podosome assembly which would in turn lead to enhanced osteoclast activity and subsequent osteopenia. These results also confirm that downstream effector proteins are likely involved in this process leading to osteopenia, particularly downstream effectors and metabolic intermediates which ultimately lead to cytoskeletal reorganization in the activated osteoclast. It should also be apparent that an aberrant BTK protein may also affect transcriptional activity in the osteoclast, as a BTK with an altered pattern of post translational modification is likely to be targeted to a different subcellular compartment. This is further supported by the ability of BTK to activate NF-κB, a potent modulator of cellular transcriptional activity.

While the invention has been described in connection with specific embodiments therefore, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims. All references cited herein are expressly incorporated in their entirety.

What is claimed is:

1. An assay for identifying a compound that modulates the activity of Bruton's Tyrosine Kinase, comprising:
(1) providing a cell expressing Bruton's Tyrosine Kinase;
(2) contacting said cell expressing Bruton's Tyrosine Kinase with a test compound; and
(3) determining whether said test compound modulates the activity of Bruton's Tyrosine Kinase.

2. The assay of claim 1, wherein said assay is a cell-based assay.

3. The assay of claim 1, wherein said assay is a cell-free assay.

4. The assay of claim 3, wherein said cell-free assay is a ligand-binding assay.

5. The assay of claim 1, wherein said test compound modulates the activity of Bruton's Tyrosine Kinase.

6. The assay of claim 1, wherein said test compound is a Bruton's Tyrosine Kinase antagonist.

7. The assay of claim 1, wherein said test compound is a Bruton's Tyrosine Kinase agonist.

8. The assay of claim 1, wherein said test compound binds to Bruton's Tyrosine Kinase.

9. The assay of claim 1, wherein said assay is for identifying compounds which will be useful for the treatment of osteoporosis.

10. A method for the treatment of osteoporosis, comprising administering to a patient in need thereof a therapeutically effective amount of a compound which was identified by the assay of claim 1.

11. A method for the treatment of osteoporosis, comprising:
(1) identifying a patient suffering from osteoporosis; and
(2) administering to said patient a therapeutically effective amount of a modulator of Bruton's Tyrosine Kinase.

12. The method of claim 11, wherein said patient is identified as suffering from osteoporosis by measuring the expression level of Bruton's Tyrosine Kinase in said patient.

13. The method of claim 11, wherein said modulator is a Bruton's Tyrosine Kinase antagonist.

14. A method for the prevention of osteoporosis, comprising:
(1) identifying a patient at risk for osteoporosis; and
(2) administering to said patient a therapeutically effective amount of a modulator of Bruton's Tyrosine Kinase.

15. The method of claim 14, wherein said patient is identified as being at risk for osteoporosis by measuring the expression level of Bruton's Tyrosine Kinase in said patient.

16. The method of claim 14, wherein said modulator is a Bruton's Tyrosine Kinase antagonist.

17. A method of decreasing the differentiation of osteoclast precursor cells into osteoclast cells, comprising contacting said osteoclast precursor cells with a Bruton's Tyrosine Kinase modulator.

18. The method of claim 17, wherein said modulator is a Bruton's Tyrosine Kinase antagonist.

19. A compound capable of modulating the activity of Bruton's Tyrosine Kinase.

20. The compound of claim 19, wherein said compound is identified by:
(1) providing a cell expressing Bruton's Tyrosine Kinase;
(2) contacting said cell expressing Bruton's Tyrosine Kinase with said compound; and
(3) determining whether said compound modulates the activity of Bruton's Tyrosine Kinase.

21. The compound of claim 19, wherein said compound is a Bruton's Tyrosine Kinase antagonist.

22. The compound of claim 19, wherein said compound is a Bruton's Tyrosine Kinase agonist.

23. The compound of claim 19, wherein said compound binds to Bruton's Tyrosine Kinase.
